(19) United States
(12) Patent Application Publication
(10) Pub. No.: US 2024/0279706 A1
(43) Pub. Date: Aug. 22, 2024

(54) METHOD TO PRODUCE RETINYL ACETATE

(71) Applicant: DSM IP ASSETS B.V., Heerlen (NL)

(72) Inventors: Peter Louis HOUSTON, Kaiseraugst (CH); Valmik Kanubhai VYAS, Kaiseraugst (CH); Anna SYMBOR-NAGRABSKA, Kaiseraugst (CH); Reed Chadbourne DOTEN, Kaiseraugst (CH); Christopher Mark FARRELL, Kaiseraugst (CH); Ethan LAM, Kaiseraugst (CH)

(21) Appl. No.: 18/292,227

(22) PCT Filed: Jul. 27, 2021
(86) PCT No.: PCT/EP2021/070909

§ 371 Date: Jan. 25, 2024

(51) Int. Cl. C12P 23/00; C12N 1/16; C12N 9/10

(52) U.S. Cl. CPC C12P 23/00; C12N 1/16; C12N 9/1029

(57) ABSTRACT

The present invention is related to a novel process for production of retinyl acetate in a host cell, particularly oleaginous yeast such as e.g. Yarrowia, wherein the product purity could be increased with reduction of unwanted side-products. Particularly, the novel process comprises fermentation in the presence of ethanol, such as e.g. in a fed-batch fermentation process. Such process is especially useful in a biotechnological process for production of vitamin A.

The present invention is related to a novel process for production of retinyl acetate in a host cell, particularly oleaginous yeast such as e.g. Yarrowia, wherein the product purity could be increased with reduction of unwanted side-products. Particularly, the novel process comprises fermentation in the presence of ethanol, such as e.g. in a fed-batch fermentation process. Such process is especially useful in a biotechnological process for production of vitamin A.

Current chemical production methods for retinoids, including vitamin A and precursors thereof, have some undesirable characteristics such as e.g. high-energy consumption, complicated purification steps and/or undesirable by-products. Therefore, over the past decades, other approaches to manufacture retinoids, including vitamin A and precursors thereof, comprising microbial conversion steps have been investigated, which would lead to more economical as well as ecological vitamin A production.

In general, the biological systems that produce retinoids are industrially intractable and/or produce the compounds at such low levels that commercial scale isolation is not practical. The most limiting factors include instability of intermediates in such biological systems and/or the relatively high production of by-products, such as e.g. fatty acid retinyl esters (FAREs), particularly using oleaginous host cells grown on vegetable oils or glucose as carbon source.

Whereas instability could be solved via expression of highly specific acetylating enzymes (ATFs) in the host cell resulting in increased accumulation of retinyl acetate, a relatively high percentage of retinol is still “lost” for vitamin A production, i.e. converted into undesired side-products including FAREs, that are difficult to purify and do not form a crystal, but instead form a wax. Thus, high concentrations of FAREs limit large scale, industrial production of pure products.

Thus, there is a strong need to improve purity and/or productivity in production of retinyl acetate using yeast, particularly oleaginous host cells such as Yarrowia.

Surprisingly, we now found that production of retinoids is improved via fermentation in the presence of ethanol, particularly in a fed-batch fermentation process, leading to reduction or abolishment of impurities including FAREs, increase in retinyl acetate and/or total retinoids.

Particularly, the present invention is directed to a process for production of retinyl acetate in a fungal host cell, preferably oleaginous yeast cell such as e.g. Yarrowia, comprising cultivation of the host cell in the presence of ethanol added during the fermentation, particularly in a fed-batch fermentation process, wherein the formation of by-products including FAREs is reduced or abolished, preferably reduced by about 50-100% based on total retinoids, compared to fermentation without ethanol, particularly compared to a (fermentation) process in the presence of triglycerides, particularly vegetable oil, as defined herein.

As used herein, the term “by-products” and “side-products” in connection with fermentative production of retinoids that are used interchangeably herein means retinoids generated during the fermentation process, with the exception of retinyl acetate. The formation of by-products which is to be reduced or abolished during the fermentation includes particularly the formation of FARE, optionally furthermore presence of retinol or retinal present in the cultivation medium and which might be purified from the retinyl acetate.

As used herein, a fermentation “in the “presence of ethanol” or “ethanol added during the fermentation” particularly refers to a fed-batch fermentation process, wherein the batch phase comprises a concentration of about 5% ethanol (v/v) or less, such as e.g. an amount of about 5, 4.5, 4, 3.5, 3, 2.5, 2, 1.5, 1% added to the cultivation medium during the batch phase of the fermentation. Ethanol feed is controlled via measurement of the pH and/or DO as known in the art, typically with feeding of about 100% ethanol and with DO setpoint of about 40 to 20% Optionally, either the batch phase or the feed further comprises glucose, such as e.g. an amount of about 20, 15, 10, 8, 5, 3, 2% or less glucose added to the cultivation medium during the batch phase of the fermentation or ratios of ethanol to glucose in the range of about 9:1, 8:2, 7:3.

As used herein, a fermentation “in the “presence of triglycerides, particularly vegetable oil” or “triglycerides, particularly vegetable oil, added during the fermentation” particularly refers to a fed-batch fermentation process, wherein the batch phase comprises a concentration of about 10% triglycerides (v/v) or less, particularly vegetable oil, such as e.g. an amount of about 10, 8, 6, 5, 4, 3, 2, 1% added to the cultivation medium during the batch phase of the fermentation. The oil to be used is particularly vegetable oil, such as e.g. oil originated from corn, soy, olive, sunflower, canola, cottonseed, rapeseed, sesame, safflower, grapeseed or mixtures thereof, including the respective free fatty acids, such as e.g. oleic acid, palmitic acid or linoleic acid.

In one preferred embodiment, the present invention is related to a process as described herein for reducing FARE-production in retinyl acetate production process, wherein the percentage of by-products including FAREs could be reduced by about 100%, i.e. more or less abolished, said process comprising the fermentation in the presence of ethanol as defined herein. Compared to a fermentation process in the presence of triglycerides as defined herein, the formation of FAREs could be reduced by at least about 50%, such as e.g. about 55, 60, 65, 70, 75, 80, 85, 90, 95 or 100%, i.e. abolishment of FARE formation.

Preferably, the present invention is directed to a retinyl acetate production process as defined herein, wherein the production of FARE formed during the fermentation is less than about 25%, such as e.g. less than about 20, 15, 10, 8, 5, 3, 2, 1% based on total retinoids.

Furthermore, the present invention is directed to a process for production of retinyl acetate in a fungal host cell, preferably oleaginous yeast cell such as e.g. Yarrowia, said process comprising fermentation in the presence of ethanol as defined herein, wherein the percentage of retinyl acetate formation could be increased by at least about 25%, such as e.g. by about 30, 35, 40, 45, 50, 55, 60, 65, 70, 75, 80, 85, 90, 95, 100% or more based on total retinoids, compared to the respective process in the absence of ethanol, particularly compared to a fermentation process in the presence of triglycerides as defined herein.

Thus, in one particular embodiment, the present invention is related to a process as described herein for production of retinyl acetate in a fungal host cell as defined herein, wherein the percentage of retinyl acetate formed during the fermentation is in the range of at least about 50-80%, such as e.g. a percentage of at least about 50, 55, 60, 65, 70, 75, 80, 85, 87, 90, 95, 98% or up to 100% based on total retinoids, said process comprising the fermentation in the presence of ethanol as defined herein.

In a further aspect, the present invention is directed to a process for increasing total retinoids, with an increase in retinoids in the range of at least about 30%, such as e.g. an increase of about 30, 35, 40, 45, 50, 60, 70, 80% or more, said process comprising the fermentation in the presence of ethanol as defined herein, as compared to the respective process in the absence of ethanol, particularly compared to a fermentation process in the presence of triglycerides as defined herein.

In one embodiment, the present invention is directed to a process as defined herein, wherein a retinyl acetate producing host cell, preferably oleaginous yeast cell such as e.g. Yarrowia, is cultivated under suitable culture conditions comprising fermentation in the presence of ethanol as defined herein, wherein the percentage of FAREs formed during the fermentation is in the range of less than 25%, preferably less than about 10%, and the percentage of retinyl acetate is the range of at least about 50-80%, preferably in the range of at least about 70, 80, 85, 87, 90, 95, 98% based on total retinoids. Compared to a process in the absence of ethanol during the fermentation, particularly wherein the host cell is cultivated in a fermentation process in the presence of triglycerides, particularly vegetable oil, as defined herein, the percentage of retinyl acetate might be increased by about 100% or more and the percentage of FAREs might be reduced to a range of about 10 to 1%, or even to less than about 1%, or even abolished, based on total retinoids.

The present invention is directed to the use of a retinyl acetate producing host cell, particularly fungal host cell, preferably oleaginous yeast cell such as e.g. such as e.g. Rhodosporidium, Lipomyces or Yarrowia, preferably Yarrowia, more preferably Yarrowia lipolytica, in a process as defined herein, e.g. in a fermentation process comprising addition of ethanol during the fermentation as defined herein, wherein the process is particularly a fed-batch fermentation, wherein the batch phase comprises a concentration of about 5% ethanol (v/v) or less, particularly 2 to 1% (v/v) ethanol in the batch phase. Particularly, suitable host cells are expressing genes coding for heterologous enzymes EC class [EC 2.3.1.84] catalyzing the enzymatic conversion of retinol into retinyl acetate. Suitable strains expressing such ATFs are described in e.g. WO2019058001 or WO2020141168.

Preferably, the host cell to be used in the present invention is expressing a heterologous ATF, particularly fungal ATF, comprising a highly conserved partial amino acid sequence of at least 7 amino acid residues selected from [NDEHCS]-H-x(3)-D-[GA] (motifs are in Prosite syntax, as defined in https://prosite.expasy.org/scanprosite/scanprosite_doc.html), wherein “x” denotes an arbitrary amino acid and with the central histidine being part of the enzyme's binding pocket, preferably wherein the 7 amino acid motif is selected from [NDE]-H-x(3)-D-[GA], more preferably selected from [ND]-H-x(3)-D-[GA], most preferably selected from N—H-x(3)-D-[GA] corresponding to position N218 to G224 in the polypeptide according to SEQ ID NO:18. Examples of such enzymes might be particularly selected from L. mirantina, L. fermentati, S. bayanus, or W. anomalus, such as e.g. LmATF1 according to SEQ ID NO:18, SbATF1, LffATF1, LfATF1, Wa1ATF1 or Wa3ATF1 as disclosed in WO2019058001, more preferably said ATFs comprising one or more amino acid substitution(s) in a sequence with at least about 20%, such as e.g. 25, 30, 35, 40, 45, 50, 55, 60, 65, 70, 75, 80, 85, 90, 92, 95, 97, 98, 99% or up to 100% identity to SEQ ID NO:18, wherein the one or more amino acid substitution(s) are located at position(s) corresponding to amino acid residue(s) selected from the group consisting of position 68, 69, 72, 73, 171, 174, 176, 178, 291, 292, 294, 301, 307, 308, 311, 312, 320, 322, 334, 362, 405, 407, 409, 480, 483, 484, 490, 492, 520, 521, 522, 524, 525, 526 and combinations thereof and as particularly exemplified in Table 4 of WO2020141168, most preferably comprising one or more amino acid substitution(s) on positions corresponding to amino acid residue(s) 69, 407, 409, 480, 484, and combinations thereof in SEQ ID NO:18.

In one particular embodiment, the host cell to be used for the process according to the present invention comprises an amino acid substitution at a position corresponding to residue 69 in the polypeptide according to SEQ ID NO:18 leading to asparagine, serine or alanine at said residue, such as e.g. via substitution of histidine by asparagine (H69N), serine (H69S) or alanine (H69A), with preference for H69A. Said modified enzyme might be originated from yeast, such as e.g. L. mirantina, L. fermentati, W. anomalus or S. bayanus, preferably from L. mirantina, optionally being combined with amino acid substitution at a position corresponding to residue 407 in the polypeptide according to SEQ ID NO:18 leading to isoleucine at said residue, such as e.g. via substitution of valine by isoleucine (V407I), optionally being combined with an amino acid substitution at a position corresponding to residue 409 in the polypeptide according to SEQ ID NO:18 leading to alanine at said residue, such as e.g. via substitution of glycine by alanine (G409A), optionally being combined with amino acid substitution at a position corresponding to residue 480 in the polypeptide according to SEQ ID NO:18 leading to glutamic acid, lysine, methionine, phenylalanine or glutamine at said residue, such as e.g. via substitution of serine by glutamic acid (S480E), lysine (S480L), methionine (S480M), phenylalanine (S480F) or glutamine (S480Q), optionally being combined with amino acid substitution at a position corresponding to residue 484 in the polypeptide according to SEQ ID NO:18 leading to leucine at said residue, such as e.g. via substitution of isoleucine by leucine (I484L). Said modified enzyme might be originated from yeast, such as e.g. L. mirantina, L. fermentati, W. anomalus or S. bayanus, preferably from L. mirantina. In a most preferred embodiment, the ATF to be used for the process according to the present invention is a modified ATF comprising amino acid substitutions S480Q_G409A_V407I_H69A_I484 L and is obtainable from Lachancea mirantina.

As used herein, the term “host cell” includes retinyl-acetate producing cells, i.e. capable of synthesizing retinol and expressing ATF as defined herein resulting in retinyl acetate with a percentage as defined herein based on total retinoids produced by said host cell. Optionally, such host cell is furthermore capable of producing carotenoids. A “fungal host cell” particularly includes yeast cells, i.e. retinyl acetate-producing yeast cells, including but not limited to Yarrowia, Rhodosporidium, or Lipomyces.

Optionally, the host cell, such as e.g. Yarrowia, capable of producing retinyl acetate from conversion of retinol, is expressing further enzymes used for biosynthesis of beta-carotene and/or additionally used for catalyzing conversion of beta-carotene into retinal and/or retinal into retinol. The skilled person knows which genes to be used/expressed for either biosynthesis of beta-carotene and/or bio-conversion of beta-carotene into retinol. Such host cell further being capable of expressing ATF genes as defined herein and/or further genes required for biosynthesis of vitamin A, is cultured in an aqueous medium comprising addition of ethanol during the fermentation, optionally supplemented with appropriate nutrients under aerobic or anaerobic conditions and as known by the skilled person to enable production of retinyl acetate. Preferably, the fermentation is performed in fed-batch, wherein the batch phase comprises a concentration of about 5% or less ethanol, particularly 2 to 1% ethanol, and with feed of 100% ethanol as particularly exemplified herein. Particularly, fermentations are run in fed-batch stirred tank reactors. Fermentations can be run for 5 to 14 days, such as e.g. for around 118 h. Fermentation products including retinyl acetate may be harvested from the cultivation at a suitable moment, e.g. when the tank fills due to addition of the feed. Depending on the host cell, preferably, production of retinoids such as e.g. vitamin A, precursors and/or derivatives thereof such as retinal, retinol, retinyl acetate, particularly retinyl acetate, can vary, as it is known to the skilled person. The retinoids including but not limited to retinol, retinyl acetate, vitamin A might be used as ingredients/formulations in the food, feed, pharma or cosmetic industry. Cultivation and isolation of beta-carotene and retinoid-producing host cells selected from Saccharomyces is described in e.g. WO2008042338.

In one embodiment, the host cell to be used for the process according to the present invention might comprise further modifications, such as modification in endogenous enzyme activities leading to conversion of retinol into FAREs. Particularly, such modifications include deletion of endogenous lipase activities, i.e. activities in enzymes involved in pre-digestion of triglyceride oils such as e.g. vegetable oil into glycerol and fatty acids that are normally expressed in oleaginous host cells. Suitable enzymes to be modified in a host cell used in the process as defined herein might be selected from endogenous enzymes belonging to EC class 3.1.1.-, including, but not limited to one or more enzyme(s) with activities corresponding to Yarrowia LIP2, LIP3, LIP4, LIP8, TGL1, LIP16, LIP17, or LIP18 activities, preferably reduction or abolishment of endogenous genes encoding enzymes with activities corresponding to Yarrowia LIP2 and/or LIP3 and/or LIP4 and/or LIP8 activities.

As used herein, an enzyme having activity corresponding to the respective LIP activity in Yarrowia includes not only the genes originating from Yarrowia, e.g. Yarrowia lipolytica, such as e.g. Yarrowia LIP2, LIP3, LIP4, LIP8, TGL-1, LIP16, LIP17, LIP18 or combinations thereof according to SEQ ID NOs:1, 3, 5, 7, 9, 11, 13, 15 but also includes enzymes having equivalent enzymatic activity but are originated from another source organism, particularly retinyl acetate-producing oleaginous host cell. Preferably, the host cell comprises deletion of endogenous lipase activities corresponding to Yarrowia lipolytica lipase activities 2, 3, 4, 8, or combinations thereof, in particular wherein the host cell is Yarrowia lipolytica comprising deletion of endogenous lipase activities such as lip8 or combinations of lip8, such as combination of lip8 with lip2, combination of lip8 with lip2 and lip3, or combination of lip8 with lip2, lip3, and lip4 activities.

The generation of a mutation into nucleic acids or amino acids, i.e. mutagenesis, may be performed in different ways, such as for instance by random or side-directed mutagenesis, physical damage caused by agents such as for instance radiation, chemical treatment, or insertion of a genetic element. The skilled person knows how to introduce mutations.

The terms “sequence identity”, “% identity” are used interchangeable herein. For the purpose of this invention, it is defined here that in order to determine the percentage of sequence identity of two amino acid sequences or of two nucleic acid sequences, the sequences are aligned for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/bases or amino acids. The sequence identity is the percentage of identical matches between the two sequences over the reported aligned region. The percent sequence identity between two amino acid sequences or between two nucleotide sequences may be determined using the Needleman and Wunsch algorithm for the alignment of two sequences (Needleman, S. B. and Wunsch, C. D. (1970) J. Mol. Biol. 48, 443-453). Both amino acid sequences and nucleotide sequences can be aligned by the algorithm. The Needleman-Wunsch algorithm has been implemented in the computer program NEEDLE. For the purpose of this invention the NEEDLE program from the EMBOSS package was used (version 2.8.0 or higher, EMBOSS: The European Molecular Biology Open Software Suite (2000) Rice, Longden and Bleasby, Trends in Genetics 16, (6) pp 276-277, http://emboss.bioinformatics.nl/). For protein sequences EBLOSUM62 is used for the substitution matrix. For nucleotide sequence, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

After alignment by the program NEEDLE as described above the percentage of sequence identity between a query sequence and a sequence of the invention is calculated as follows: number of corresponding positions in the alignment showing an identical amino acid or identical nucleotide in both sequences divided by the total length of the alignment after subtraction of the total number of gaps in the alignment. The identity as defined herein can be obtained from NEEDLE by using the NOBRIEF option and is labeled in the output of the program as “longest identity”. If both amino acid sequences which are compared do not differ in any of their amino acids, they are identical or have 100% identity.

The enzymes as defined herein to be expressed in a suitable host cell to be used in the present invention also encompass enzymes carrying (further) amino acid substitution(s) which do not alter enzyme activity, i.e. which show the same properties with respect to the enzymes defined herein. Such mutations are also called “silent mutations”, which do not alter the (enzymatic) activity of the enzymes according to the present invention.

With regards to the present invention, it is understood that organisms, such as e.g. microorganisms, fungi, algae or plants also include synonyms or basonyms of such species having the same physiological properties, as defined by the International Code of Nomenclature of Prokaryotes or the International Code of Nomenclature for algae, fungi, and plants (Melbourne Code). Thus, for example, strain Lachancea mirantina is a synonym of strain Zygosaccharomyces sp. IFO 11066, originated from Japan.

The present invention is directed to a process for production of retinyl acetate, wherein the retinyl acetate is generated via acetylation of retinol (particularly at least 65% as trans-retinol) as disclosed herein by the action of modified/non-modified ATF as described herein, wherein the acetylating enzymes are heterologous expressed in a suitable host cell under suitable conditions as described herein and wherein the host cell is cultivated in a medium comprising an effective amount of ethanol added during the fermentation. The produced retinyl acetate might be isolated and optionally further purified from the medium and/or host cell. Said acetylated retinoids defined herein can be used as building blocks in a multi-step process leading to vitamin A. Vitamin A might be isolated and optionally further purified from the medium and/or host cell as known in the art.

As used herein, the term “specific activity” or “activity” with regards to enzymes means its catalytic activity, i.e. its ability to catalyze formation of a product from a given substrate. The specific activity defines the amount of substrate consumed and/or product produced in a given time period and per defined amount of protein at a defined temperature. Typically, specific activity is expressed in μmol substrate consumed or product formed per min per mg of protein. Typically, μmol/min is abbreviated by U (=unit). Therefore, the unit definitions for specific activity of μmol/min/(mg of protein) or U/(mg of protein) are used interchangeably throughout this document. An enzyme is active, if it performs its catalytic activity in vivo, i.e. within the host cell as defined herein or within a suitable (cell-free) system in the presence of a suitable substrate. The skilled person knows how to measure enzyme activity. Analytical methods to evaluate the capability of a suitable ATF (wild-type or modified) as defined herein for retinyl acetate production, i.e. acetylation of retinol, or enzymes with lipase activity as defined herein are known in the art, such as e.g. described in Example 4 of WO2014096992. In brief, titers of products such as retinyl acetate, retinol, trans-retinal, cis-retinal, beta-carotene and the like can be measured by HPLC.

With regards to suitable host cells comprising specific enzymes involved in biosynthesis of beta-carotene and that are expressed and active in vivo leading to production of carotenoids, e.g. beta-carotene, both genes and methods to generate carotenoid-producing host cells are known in the art, see e.g. WO2006102342. Depending on the carotenoid to be produced, different genes might be involved.

As used herein, a “retinol-producing host cell” is a host cell, wherein the respective polypeptides are expressed and active in vivo, leading to production of retinoids, e.g. vitamin A and its precursors including retinol, via enzymatic activity of the ATFs as described herein. The genes of the vitamin A pathway and methods to generate retinoid-producing host cells are known in the art. The term retinoid includes retinol, which is used as a substrate for the modified acetylating enzymes as defined herein. A “retinyl acetate-producing host cell” is the respective host cell capable of acetylation of retinol into retinyl acetate.

Retinoids as used herein include beta-carotene cleavage products also known as apocarotenoids, including but not limited to retinal, retinolic acid, retinol, retinoic methoxide, retinyl acetate, retinyl esters, 4-keto-retinoids, 3 hydroxy-retinoids or combinations thereof. Biosynthesis of retinoids is described in e.g. WO2008042338.

“Retinal” as used herein is known under IUPAC name (2E,4E,6E,8E)-3,7-Dimethyl-9-(2,6,6-trimethylcyclohexen-1-yl)nona-2,4,6,8-tetraenal. It is herein interchangeably referred to as retinaldehyde or vitamin A aldehyde and includes both cis- and trans-isoforms, such as e.g. 11-cis retinal, 13-cis retinal, trans-retinal and all-trans retinal.

The term “carotenoids” as used herein is well known in the art. It includes long, 40 carbon conjugated isoprenoid polyenes that are formed in nature by the ligation of two 20 carbon geranylgeranyl pyrophosphate molecules. These include but are not limited to phytoene, lycopene, and carotene, such as e.g. beta-carotene, which can be oxidized on the 4-keto position or 3-hydroxy position to yield canthaxanthin, zeaxanthin, or astaxanthin. Biosynthesis of carotenoids is described in e.g. WO2006102342.

“Vitamin A” as used herein may be any chemical form of vitamin A found in solutions, in solids and formulations, and includes retinol, retinyl acetate and retinyl esters. It also includes retinoic acid, such as for instance undissociated, in its free acid form or dissociated as an anion.

It was also surprisingly found that the production of other isoprenoid-derived products, such as steviol glycosides, was improved via fermentation of a recombinant fungal host cell, particularly a yeast cell such as e.g. a Saccharomyces cerevisiae cell, more particularly an oleaginous yeast cell such as e.g. a recombinant Yarrowia cell (suitably modified to be able to produce steviol glycosides), in the presence of ethanol. It was found that the production of said products was increased when ethanol was provided as a carbon source, particularly in a fed-batch fermentation process, when compared to the respective process in the absence of ethanol. More particularly, the production of said products was increased with the use of pure ethanol feeds or mixed ethanol feeds (e.g. ethanol/glucose mixture) following the batch phase of growth.

Accordingly, the present disclosure is directed to a process for the production of steviol glycosides in a recombinant fungal host cell, preferably a yeast cell such as e.g. a Saccharomyces cerevisiae cell, more preferably an oleaginous yeast cell such as e.g. a recombinant Yarrowia cell, more preferably Yarrowia lipolytica (suitably modified to be able to produce steviol glycosides), said process comprising fermentation in the presence of ethanol as defined herein. The present disclosure is also directed to the use of a host cell that produces steviol glycosides, preferably a yeast cell such as e.g. a Saccharomyces cerevisiae cell, more preferably an oleaginous yeast cell such as e.g. a recombinant Yarrowia cell, more preferably Yarrowia lipolytica (suitably modified to be able to produce steviol glycosides), in a fermentation process comprising addition of ethanol during the fermentation as defined herein.

Suitable fungal host cells for the production of steviol glycosides are known in the art and are described in e.g. WO2011153378, WO2013022989, WO2014122227, WO2013110673, and WO2015007748.

The produced steviol glycosides might be isolated and optionally further purified from the medium and/or host cell according to a method known to the skilled person in the art.

The following examples are illustrative only and are not intended to limit the scope of the invention in any way. The contents of all references, patent applications, patents, and published patent applications, cited throughout this application are hereby incorporated by reference, in particular WO2019058001, WO2020141168, WO2008042338, WO2014096992, WO2006102342, WO2016172282, WO2011153378, WO2013022989, WO2014122227, WO2013110673, and WO2015007748.

### EXAMPLES

### Example 1: General Methods and Plasmids

All basic molecular biology and DNA manipulation procedures described herein are generally performed according to Sambrook et al. (eds.), Molecular Cloning: A Laboratory Manual. Cold Spring Harbor Laboratory Press: New York (1989) or Ausubel et al. (eds). Current Protocols in Molecular Biology. Wiley: New York (1998).

DNA transformation. Strains were transformed by overnight growth on YPD plate media; 50 μl of cells was scraped from a plate and transformed by incubation in 500 μl with 1 μg transforming DNA, typically linear DNA for integrative transformation, 40% PEG 3550MW, 100 mM lithium acetate, 50 mM Dithiothreitol, 5 mM Tris-Cl pH 8.0, 0.5 mM EDTA for 60 minutes at 40° C. and plated directly to selective media or in the case of dominant antibiotic marker selection the cells were out grown on YPD liquid media for 4 hours at 30° C. before plating on the selective media. URA3 marker recycling was performed using 5-fluoroorotic acid (FOA). Episomal hygromycin resistance marker plasmids were cured by passage on non-selective media, with identification of Hyg-sensitive colonies by replica plating colonies from non-selective media to hygromycin containing media (100 μg/mL).

DNA molecular biology. Plasmids MB9523 containing expression systems for DrBCO, LmATF-S480Q_G409A_V407I_H69A_I484 L, and FfRDH (SEQ ID NO:17) was synthesized at Genscript (Piscataway, NJ, USA). Plasmid MB9523 contains the ‘URA3’ for marker selection in Yarrowia lipolytica transformations. For gene insertion by random nonhomologous end joining of the gene and marker, Sfil-digested MB9523 plasmid fragment of interest was purified by gel electrophoresis and Qiagen gel purification column. Clones were verified by sequencing. Typically, genes are synthesized by a synthetic biology at GenScript (Piscataway, NJ).

Plasmid list. Plasmid, strains, nucleotide and amino acid sequences that were used are listed in Table 1, 2 and the sequence listing. In general, all non-modified sequences referred to herein are the same as the accession sequence in the database for reference strain CLIB122 (Dujon B, et al, Nature. 2004 Jul. 1; 430(6995):35-44).

TABLE 1
list of plasmids used for construction of the
strains for overexpression or deletion of the
respective genes indicated as ″insert″. ″LmATF1-
mut″ refers to Lachancea mirantina (LmATF1; SEQ
ID NO: 13 in WO2019058001) carrying aa sub-
stitutions S480Q_G409A_V407I_H69A_1484L. ″DrBCO″
refers to BCO originated from Danio rerio (see
SEQ ID NO: 16 in WO2020141168); ″FfRDH″ refers
to RDH originated from Fusarium fujikuroi (see
SEQ ID NO: 22 in WO2020141168). For more
explanation, see text.

| | | | SEQ |
|---|---|---|---|
| Plasmid | Insert | Marker | ID NO |
| MB9523 | DrBCO; LmATF1-mut; FfRDH | URA3 | 17 |

TABLE 2
list of Yarrowia lipolytica strains used. Construction of ML17544 is
described in Table 2 of WO2020141168. For more details, see text.

| Strain | Description |
|---|---|
| ML18812 | ML17544 transformed with MB9523 |

Fermentation conditions. Fed-batch fermentations were identical to the previously described conditions except using Drakeol 5 (Penreco, Karns City, PA, USA) or another overlay and stirred tank that was corn oil, glucose or ethanol fed in a bench top reactor with 0.5 L to 5 L total volume (see WO2016172282). The batch medium carbon source composition and feed medium are listed in Table 3. Feeding was initiated after the initial batch carbon had been consumed, with feed added in a controlled manner to maintain a dissolved oxygen level (DO) setpoint.

Briefly, the fermentations were run in 3.0 L flood volume in glass New Brunswick or Eppendorf fermentation systems. The fermentor was batched with the following components: 2228 mL of deionized water, MgSO₄·7H₂O is 1.96 g/kg and NaCl is 0.20 g/kg, 10.46 mL, 1.04 g CaCl₂)-2H₂O, 26.18 g (NH₄)2SO₄, 27.10 g KH₂PO₄, 19.62 g Tastone yeast extract (Marcor, Leominster, MA), 26 mL DF204 antifoam, 0.654 μL, thiamine HCl at 4 mg/ml, trace elements stock solution 3.27 ml/L 20) containing: 200 g/kg citric acid, 27.3 g/kg FeSO₄·7H₂O, 19.6 g/kg Na₂MoO₄·2H₂O, 18.7 CuSO₄·5H₂O, 4.9 H₃BO₃, 21.9 MnSO₄·H₂O, 30.2 ZnSO₄·7H₂O, and autoclaved. After cooling a carbon source was added along with 800 mL Drakeol 5 or other second phase. The fermentation was inoculated with 200 ml overnight shake flask cultures of YP media grown with 250 RPM agitation at 30° C. and the specific carbon sources shown in Table 3. Fermentation parameters were agitation at 1000 RPM, airflow at 4.6 LPM for ethanol and 2.3 LPM for oil and mixed fatty acids, pH controlled at 5.5 control with NH₄OH, and the temperature set to 30° C. At feed start, feed was added to maintain the DO setpoint at 40%. The DO setpoint was ramped down to 20% in a linear fashion over the following 24 hours by increased feed rate. The DO was then maintained at 20% via feed addition for the remainder of the fermentation.

TABLE 3
fermentation feeding protocol. Fermentation 2^{nd} phase was always
~20% by weight Drakeol 5. For more explanation, see text.

| Run: | Batch: | Feed: |
|---|---|---|
| ethanol | 5% glucose (w/v) | 100% ethanol (w/v) |
| | 1% ethanol (w/v) | |
| oil | 6.25% oleic acid (v/v) | 100% corn oil (w/v) |
| | 1.4% corn oil (v/v) | |

Retinoid quantification. Analysis of retinoids were carried out with a C4 reverse phase retinoid method (see below) and C18 as described elsewhere (WO2020141168). The addition of all added intermediates gives the total amount of retinoids.

C4 reverse phase chromatography. For exact determination of discrete retinoids the long run reverse phase system was used. We separated analytes at 230 nm and 325 nm through the Agilent 1290 instrument with YMC Pro C4, 150×3.0 mm 3 μm column (YMC America, Devens, MA) stationary phase, and a 5 μl injection loop volume and column and sample tray controlled at 23° C. with gradients described in Table 4B. Analytes were detected at 230 nm and 325 nm and the peaks identity verified with LCMS. The analytes separated as discrete peaks that were assigned according to Table 4A.

TABLE 4A
list of analytes using C4-reverse phase method. The addition
of all added intermediates gives the total amount retinoids.
“RT” means retention time. For more details, see text.

| Intermediates | RT [min] | λ max [nm] |
|---|---|---|
| trans-retinol | 20.21 | 325 |
| cis-retinol | 20.32 | 325 |
| dihydro-retinol | 20.75 | 290 |
| trans-retinal | 20.89 | 380 |
| cis-retinal | 21.02 | 380 |
| trans-retinyl-acetate | 22.15 | 325 |
| cis-retinyl-acetate | 22.35 | 325 |
| dihydro-retinyl acetate | 22.60 | 290 |
| retinyl esters | 26.30 | 325 |

TABLE 4B
UPLC Method Gradient with solvent A: acetonitrile; solvent
B: water; solvent C: water/acetonitrile/methanesulfonic
acid 1000:25:1. For more details, see text.

| Time | | | | Flow |
|---|---|---|---|---|
| [min] | % A | % B | % C | [ml/min] |
| 0 | 5 | 85 | 10 | 0.5 |
| 20 | 98 | 0 | 2 | 0.5 |
| 35 | 98 | 0 | 2 | 0.5 |
| 35.1 | 5 | 85 | 10 | 0.5 |
| 40 | 5 | 85 | 10 | 0.5 |

Method Calibration. Method is calibrated using high purity retinyl acetate received from DSM Nutritional Products, Kaiseraugst, CH. Retinols and retinal are quantitated against retinyl acetate. Dilutions were prepared as follows. 40 mg of retinyl acetate was weighed into a 100 mL volumetric flask, and dissolved in ethanol, yielding a 400 μg/mL solution. This solution was sonicated as required to ensure dissolution. 5 mL of this 400 μg/mL solution was diluted into 50 mL (1/10 dilution, final concentration 40 μg/mL), 5 mL into 100 ml (1/20 dilution, final concentration 20 μg/mL), 5 ml of 40 μg/mL into 50 mL (1/10 dilution, final concentration 4 μg/mL), 5 mL of 20 μg/mL into 50 ml (1/10 dilution, 2 μg/mL), using 50/50 methanol/methyl tert-butyl ether (MTBE) as the dilutent. All dilutions were done in volumetric flasks. Purity of retinyl acetate was determined by further diluting the 400 μg/mL stock solution 100-fold (using a 2 mL volumetric pipet and a 200 mL volumetric flask) in ethanol. Absorbance of this solution at 325 nm using ethanol was taken as the blank, with adjustment of the initial concentration using the equation (Abs*dilution (100)*molecular weight (328.5)/51180=concentration in mg/ml). Because of quick out-maximization of UV absorbance of retinyl acetate, lower concentrations are better.

Sample preparation. Top second-phase layer samples from each strain were diluted at a 25-fold dilution or higher, if needed, into tetrahydrofuran (THF). Fermentation whole broth was prepared using a 2 mL Precellys (Bertin Corp, Rockville, MD) tube, adding 25 μl of well mixed broth and 975 μl of THF. Precellys (3×15×7500 rpm) for two cycles with a freeze at −80° C. for 10 minutes between cycles. Cell debris was spun down via centrifugation for 1 minute at 13000 rpm. These samples were diluted 10-fold in THF for analysis.

### Example 2: Impact of Carbon Source on Retinoid Production in Fed-Batch Fermentations Using Yarrowia

To evaluate the impact of carbon source on the production of retinoids in Yarrowia lipolytica, fermentations in the presence of ethanol or oil (see Table 3) were run, and the purity as well as percentage of retinyl acetate assessed via measuring percentage of FARE from strain ML18812.

Fermentation in the presence of ethanol resulted in a strong reduction of FARE formation compared to fermentations in the presence of corn oil. Percentage of total retinoids as well as retinyl acetate could also be increased in comparison to corn oil fermentations. The results are shown in Table 5.

TABLE 5
effect of ethanol on production of FARE, retinoids and retinyl acetate
(“retAc”) using strain ML18812. “+++” is in
the top quartile of the maximum value, “++” is in the
mid quartile and “+/−” indicates trace amounts observed,
while “−−−” indicates not observed, “retAc”
means retinyl acetate. For more explanation, see text.

| Carbon source | Total retinoids | retAc | FAREs |
|---|---|---|---|
| Ethanol | +++ | +++ | −−− |
| corn oil | ++ | +/− | +++ |

1. A process for production of retinyl acetate in a fungal host cell, particularly oleaginous yeast cell, preferably Yarrowia, said process comprising fermentation of the host cell in the presence of ethanol.

2. The process according to claim 1 wherein the fermentation is a fed-batch fermentation.

3. The process according to claim 2 comprising a concentration of about 5% or less (v/v) ethanol added to the cultivation medium during the batch phase.

4. The process according to claim 1, said process comprising fermentation of the host cell in the absence of triglycerides, preferably vegetable oil.

5. The process according to claim 1, wherein the formation of by-products including fatty acid retinyl esters (FAREs) is reduced or abolished, preferably reduced by at least about 50% based on total retinoids and compared to a process comprising fermentation of the host cell in the absence of ethanol.

6. The process according to claim 1, wherein the percentage of retinyl acetate based on total retinoids is increased, preferably by at least about 25%, compared to a process comprising fermentation of the host cell in the absence of ethanol.

7. The process according to claim 1, wherein the production of total retinoids is increased, preferably by at least about 30%, compared to a process comprising fermentation of the host cell in the absence of ethanol.

8. The process according to claim 1, wherein the host cell is transformed with and is expressing heterologous genes, preferably genes encoding acetylating enzymes catalyzing the conversion of retinol to retinyl acetate, more preferably fungal enzymes, most preferably originated from Lachancea mirantina.

9. The process according to claim 8, wherein the acetylating enzyme comprises one or more amino acid substitution(s) in a sequence with at least about 20%, such as e.g. 25, 30, 35, 40, 45, 50, 55, 60, 65, 70, 75, 80, 85, 90, 92, 95, 97, 98, 99% or up to 100% identity to SEQ ID NO:18, wherein the one or more amino acid substitution(s) are located at position(s) corresponding to amino acid residue(s) selected from the group consisting of position 68, 69, 72, 73, 171, 174, 176, 178, 291, 292, 294, 301, 307, 308, 311, 312, 320, 322, 334, 362, 405, 407, 409, 480, 483, 484, 490, 492, 520, 521, 522, 524, 525, 526 and combinations thereof in a polypeptide according to SEQ ID NO:18.

10. A process for reduction of enzymatic conversion of retinol into FAREs, preferably with a percentage of FARE based on total retinoids being less than 25%, said process comprising fermentation of a retinyl acetate producing host cell in the presence of ethanol.


SEQUENCE LISTING (18 sequences)

SEQ ID NO: 1 - protein, 334 aa, Yarrowia lipolytica
MKLSTILFTACATLAAALPSPITPSEAAVLQKRVYTSTETSHIDQESYNFFEKYARLANIGYCVGPGTKIFKPFNCGLQCAHFPNVELIEEFHDPRLIFDVSGYLAVDHASKQIYLVIRGTHSLEDVITDIRIMQAPLTNFDLAANISSTATCDDCLVHNGFIQSYNNTYNQIGPKLDSVIEQYPDYQIAVTGHSLGGAAALLFGINLKVNGHDPLVVTLGQPIVGNAGFANWVDKLFFGQENPDVSKVSKDRKLYRITHRGDIVPQVPFWDGYQHCSGEVFIDWPLIHPPLSNVVMCQGQSNKQCSAGNTLLQQVNVIGNHLQYFVTEGVCGI

SEQ ID NO: 2 - DNA, 1005 bp, Yarrowia lipolytica
atgaagctttccaccatcctcttcacagcctgcgctaccctggctgccgccctcccttcccccatcactccttctgaggccgcagttctccagaagcgagtgtacacctctaccgagacctctcacattgaccaggagtcctacaacttctttgagaagtacgcccgactcgcaaacattggatattgtgttggtcccggcactaagatcttcaagcccttcaactgtggcctgcaatgtgcccacttccccaacgttgagctcatcgaggagttccacgacccccgtctcatctttgatgtttctggttacctcgctgttgatcatgcctccaagcagatctaccttgttattcgaggaacccactctctggaggacgtcataaccgacatccgaatcatgcaggctcctctgacgaactttgatcttgctgctaacatctcttctactgctacttgtgatgactgtcttgtccacaatggcttcatccagtcctacaacaacacctacaatcagatcggccccaagctcgactctgtgattgagcagtatcccgactaccagattgctgtcaccggtcactctctcggaggagctgcagcccttctgttcggaatcaacctcaaggttaacggccacgatcccctcgttgttactcttggtcagcccattgtcggtaacgctggctttgctaactgggtcgataaactcttctttggccaggagaaccccgatgtctccaaggtgtccaaagaccgaaagctctaccgaatcacccaccgaggagatatcgtccctcaagtgcccttctgggacggttaccagcactgctctggtgaggtctttattgactggcccctgatccaccctcctctctccaacgttgtcatgtgccagggccagagcaataaacagtgctctgccggtaacactctgctccagcaggtcaatgtgattggaaaccatctgcagtacttcgtcaccgagggtgtctgtggtatctaa

SEQ ID NO: 3 - protein, 498 aa, Yarrowia lipolytica
MPLELPSLNASIVGNTVQNGAVEQFLNIRYADIPGKFEKPVLKNDWNGAEIDATKVGPVCPQPRTPFNFFSVPDDLWEKVNVDTYQDGLLCDNLIVTRPKGVSANARLPTVVWIHGGSNIEGSIYNLIYEPQFLVAESVRVGKPIVHVCIEYRLGLAGFLTKNGKGNWGTWDQYTGCQWVNRHIQDFGGDPLNVTLTGESAGSVAVHNMLIKDSMNGRKLFRNAVMMSGTLETITPQPPKWHARLEEKVAKVTGKEVADLASLSDKELLDAQIKLNVAVCMTCDDGDFFEPGWKQHLTPDWLDKLIISDCKDEGMLYFLPVNAQDDEELLAKVAKSPVGKEISELYGIKEGGDIKSACLDLKTDATFNYFNHLLFKKMEEARNNGSTSRVYRLAVDEPNPHNPDQRAHHAVDVLYMFNSTKFNEHGDKLSRLFQSHFLRLAYGLEPWDHRNFGVYRNGGYQQLPLSELNKVRPVERYEALSKMDFGQVGRLSNALSRL

SEQ ID NO: 4 - DNA, 1497 bp, Yarrowia lipolytica
atgcctctcgaactcccctcgctcaacgcctcgattgtcggcaacaccgttcagaacggcgctgttgagcagtttctcaacatccgatacgccgacattcctggcaagtttgagaagcccgtgctcaagaacgattggaacggcgcggagatcgacgccaccaaggtcggtcccgtgtgcccccaaccccgcaccccattcaacttcttctccgtgccagacgacctctgggagaaagtcaatgtggacacgtaccaggacggtctgctgtgcgacaacctgattgtgacgcgaccgaagggcgtgtctgccaacgcccggctgcccactgttgtgtggatccacggcggctccaatattgagggcagtatctacaacctcatctatgagccccagttcctggtggcagagtcggtgcgagtaggcaagccgattgtgcacgtgtgtatcgagtaccgactgggtctcgcgggcttcctcaccaagaacggcaagggcaactggggcacgtgggatcagtacacgggctgccagtgggtcaaccgccacattcaggactttggaggcgatcctttgaacgtgacattgaccggtgagtctgccggctctgtagcagtccataacatgctcatcaaggactccatgaacggccgaaagttgttccgaaatgccgtcatgatgagtggcactctcgagaccatcactcctcagcctcccaagtggcatgctcgtttggaggagaaggtggccaaggtcactggcaaggaagtggccgaccttgcttctctgtccgataaggagctgctcgacgcccagatcaagctcaatgtggctgtgtgcatgacttgcgacgacggcgactttttcgagcccggatggaagcagcatctgactcctgactggctcgacaagctcatcatctccgattgcaaggacgagggcatgctgtatttcctgccagtcaacgcgcaggacgacgaggagctgttggcaaaggtggccaagtcgcccgtgggtaaggagatttccgagctttacggcatcaaggagggtggcgatatcaagtctgcgtgtctcgatctcaagactgacgccaccttcaattactttaaccatctgctgttcaagaagatggaggaggcccgaaacaacggctccacttctcgagtttaccgtctggccgtcgatgagcccaacccccacaaccccgaccagcgggcccaccacgccgtcgacgtgctgtacatgttcaactcgaccaagttcaacgagcacggcgacaagctgtctcggctgttccagagccactttttgcggctggcgtatggcctggagccctgggaccatcgaaactttggagtgtacagaaacggcggctaccagcagctgccgctgagtgagttgaacaaggtccgacccgtcgagcggtacgaggcgctgtccaagatggactttggccaggttgggcgtttgtccaatgcgctttcgcgcctatga

SEQ ID NO: 5 - protein, 406 aa, Yarrowia lipolytica
MAGFNFTFGQVISYLASMLYGQVDATSSSTRIQATQDLYDFTAKFSRLSNIAYCINAPFTPLRTDFTCGESCRYFPDLQLDSVFGGNFSSASTTGYIAYDHKKKEKYIVFRGTFSIPDIITDIQFQTAPWLTSLPTHLIPTKEDFEHKQAILKHYAAENKGLSNLEERQDVVHEDPSLVPKKMDKCENCQIHDGFAKGFNETIEHAGPQIEKFLGNNTDYKMFVVGHSLGAAQAQLFATQFKLLGFDPYMINFGQPRLGNPEFAAYINQLWFNDTGLVVNDARRFYRVTHWNDIVVGVPDWLNYTHSIGEVFIDEESVYPKLDKVVVCEGGENPLCHRGTFNLWSRINFLQNHLAYIFYIGLCAFNIGRRDVLNMPQYQGNFSYQHNIDPNYNYDTKVPTRISKSN

SEQ ID NO: 6 - DNA, 1221 bp, Yarrowia lipolytica
atggcagggttcaacttcactttcgggcaggtgatttcgtacctggcttccatgctctatggacaggtggatgccacttcatcctccactcgaatccaggccacccaggatctgtacgacttcacagccaagttttcgcgactctcaaacatcgcgtactgcatcaatgcccccttcacgcctctcagaacggacttcacctgcggagaaagctgtcggtacttccccgacctccagctggactcagtgtttggtggtaacttctcctcagcctccactaccggctacattgcatacgaccacaagaagaaggaaaagtacattgtgtttcgaggaactttcagtatccctgatatcatcacagacattcaatttcagactgccccttggttgacctctctgcccacgcatctgatccctaccaaggaggactttgaacacaagcaggccatcctgaagcactacgctgccgaaaacaagggtctcagcaacctggaagagcgacaggatgttgtgcatgaagaccctagcctggttcccaagaaaatggacaagtgcgagaactgccagatccatgacggattcgccaagggcttcaacgagactatcgagcatgccggaccccagattgaaaagttcctgggcaataacaccgactacaagatgtttgttgttggccactctctaggagctgctcaggcccagctgtttgctacacagttcaaactgctgggatttgacccttacatgatcaactttggacagcctcgacttggaaaccctgagttcgccgcctacatcaaccagctgtggttcaacgatactggtctggttgtcaatgatgcccgacgattctaccgagtgactcactggaacgatatcgtcgtgggagtgcccgactggctcaattacacccactctatcggagaggtgttcatagacgaggagagcgtttaccccaagctggacaaggtggtggtgtgcgagggaggagagaaccccctgtgccaccgaggaactttcaacctgtggtcacgaatcaacttcctgcagaaccatttggcttatatcttctacattggtctgtgtgctttcaacattggccgaagagacgtgctcaacatgccacaataccagggcaacttctcgtaccagcacaacatcgaccccaactacaattacgataccaaggttcccacccggatcagtaaatcaaactaa

SEQ ID NO: 7 - protein, 371 aa, Yarrowia lipolytica
MVSLSARIKDFFSVLLLGAATITPSTQTAGVSQGFYDFARDFAHLSNIAYCVNAPITPLNPDFTCGNSCKHFPEIELVKTFGGNFFKTSITGYLAVDHVKKEKYVVFRGTFSLADAITDMQFQLSPFLVDVPALNTFSANDTTAEAQTHCEGCKIHDGFSKAFTETWGNIGEDLQKHLDANPDYQLYVTGHSLGAAMALLGATSIKLKGYDPILINYGQPRVGNKPFAEFINKLWFGEGNGLEITPERKLYRMTHWNDIFVGLPNWEGYTHSNGEVYINNRFINPPLKDVISCAGGENSKCYRSSFSLLSQINLLQNHLAYIDYIGYCALNIGRRELADQEHYTGPYYYGHRSEEDFKKLGLELSTPQVEN

SEQ ID NO: 8 - DNA, 1116 bp, Yarrowia lipolytica
atggtatccctctctgctcgaatcaaagactttttttcggtcctcctcctcggagctgcaaccatcactccctccacacagaccgcaggcgtgtctcaagggttctatgattttgctcgggactttgcccatctgtccaacattgcctactgtgtcaatgctcccatcactccactgaacccggacttcacctgtggcaactcgtgcaagcactttccggaaattgagcttgtgaagacatttggaggcaacttcttcaagacctccattacgggctacctggctgtcgatcatgtcaagaaggagaagtacgttgtcttccgaggaaccttttcgctggcagacgcgatcacggacatgcagttccagctgtctcctttcctggtcgatgtgcctgccctgaacactttctcagctaatgacaccaccgcagaggcccagacgcactgtgagggctgcaaaattcacgacggcttctccaaggcctttaccgagacctggggtaacattggtgaggatctgcagaaacacctggacgctaacccggactaccagctgtacgtgactggccattctctgggagctgctatggcccttcttggagctacttccatcaagctcaagggctacgatcccattctcatcaactacggacagccccgagtcggaaacaagcccttcgctgagttcattaacaagttgtggtttggagaaggcaacggtctggaaatcacccccgagagaaagctgtaccgaatgacccactggaacgacatctttgttggcctgcccaactgggagggatacacccactctaacggtgaagtatacatcaacaaccggttcatcaaccctcctctcaaggatgtcatctcttgtgctggaggcgaaaactcgaagtgctaccgatcctcgttcagcctgctgtcccagatcaatctgctccaaaaccacctggcttacattgattacattggatactgcgctctgaacattggtcgacgagagcttgccgatcaggaacattacactggtccttattactatggtcatcgatctgaggaggactttaagaagttgggcttggagctatccaccccacaagttgagaactga

SEQ ID NO: 9 - protein, 569 aa, Yarrowia lipolytica
MHVPVLGRLELSHWPMLFVSLFLVCLEFLVSFITALLPNFFIEWCHDKVEILQVYFPSTGEVALRKSPEQLETVNALLKARDFVEICDFHGYQAEEHVVQTKDGFLLGVHRILPKNPAALLTDDPEVLSTPAPPPAFLAGNLEVYSSPPKRGSKRSVSSKLITRPVVYLHHGLLMNSEVWVVNTDAKKSIAFALADLGFDVWLGNNRGNKYSRKHMKYNPESREFWDFCLDDFALFDIPDSIDYILSVTKQKSLSYIGFSQGSAQAFASLAIRPPLNDKVNLFIAVAPAMSPPGLRSKIVNSLMRASPQLLFLCFGRRAILSSSPFWESVLDAKLYARIIDAACRMLFDWYGENITWPQKIAAYHHLYSYTSVKSVVHWFQIIRAASFHMFEDVINSPLDPHLCKYSTVTRYPTENIRTPIALIYGKTDSLVDIDQMLSTLPSSTIAFGVPKHEHLDLLWGDEADTLVIPKIVALLDYYTPVPRSNKSSSDHISERLFEPANSKVSGLGLSQDLKSPLSLKKRSLSNSSSIQDSHKVGGDTDSVLSLRNLTPKGYALGSAKPVSGTLNP

SEQ ID NO: 10 - DNA, 1710 bp, Yarrowia lipolytica
atgcacgtccccgttctaggacgtctcgagctgtcacattggcccatgctgtttgtgtcgctcttcctcgtctgcctcgagtttctcgtgagcttcatcaccgctctgctccccaacttcttcattgaatggtgccacgacaaggtcgagattctgcaggtctacttccccagcaccggcgaggttgctctcagaaagtctcccgagcagctcgaaaccgtcaacgctctgctcaaggcccgagactttgttgaaatctgcgatttccatggctaccaggccgaagaacatgtggttcagaccaaggatggatttctgctaggagtgcaccgaatcctgcccaagaaccctgctgctctgctcaccgacgaccccgaagtcctcagcacccccgcccctcccccggccttcctggctggaaacctcgaagtctactcttcgccccccaaaagaggctccaagcgaagtgtgtcctccaaactgatcacccgacccgttgtctaccttcaccatggtctgctgatgaactcggaggtctgggttgtgaacacggacgccaaaaagtcgattgcttttgctctggccgacctgggatttgacgtctggctcggaaacaacagaggcaacaagtactcgcgaaaacacatgaaatacaaccccgagtcgcgcgagttctgggatttctgtctcgacgactttgcgctgtttgatatccccgattccattgactatattctgtctgtgaccaaacagaagtctctgtcttacattggattctcccagggttcagcccaggcatttgcatctctggctatccgacctcctctcaacgataaggtcaacctattcattgccgttgcccccgccatgtctcctcctggcctgagatccaagattgtcaactccctcatgagagcttctccacagctgctgttcctctgctttggtcgaagagctatcttgtcatcctctcccttctgggagtcggttctggacgcaaaactgtatgctcgaatcatcgatgctgcctgcagaatgctgtttgactggtacggagagaacatcacctggccccagaagattgcagcctaccaccacctgtactcgtacacctctgtcaagtccgtcgtgcattggttccagatcatccgagcagcctccttccacatgtttgaggatgtcatcaactctcccctggaccctcatctgtgcaagtactccactgtcacccgataccccaccgagaacattcggacacccattgctctgatctacggcaagaccgactcgctggtggacattgaccagatgctgtctactctaccctcatccactattgcatttggagtccccaaacatgagcatctcgatcttctgtggggagacgaagccgacactctggttatccccaagattgtcgctctgctcgattattacacacctgtgcccagaagtaacaagagcagcagtgaccatatcagcgagcgtctatttgagcctgctaactccaaggtctcgggtctgggactcagccaggacctcaagtcgccattgtctttgaaaaagagatctctctccaactcctcatcgatccaggactctcataaggtcggaggagacacagactcggttctaagtctccgaaacctcactcccaaggggtacgctcttggaagtgccaagccagtgagtggcactctcaacccttga

SEQ ID NO: 11 - protein, 365 aa, Yarrowia lipolytica
MLSLIAIFLLVTTALAQTANITQSTYDFVLKYGRLSNVAYCVKAPGPYELETDFTCGRSCGHFPNVTLEHQFGGDFFSTSITGFLAHDHTKKEKYIVFRGTFSLADAITDALFLQEPYLADLPPLNTTNINSTSNSARVDCPDCEIHDGFQKAYRETMVNMQGHLVAFLRNNTDYKLIVTGHSLGAATALLMGINLKNLGFDPMVITFGQPRVGNKAFADYADSLFFKQGDNGLNINPERRLYRVTHWNDIVVGVPFWSGYTHTLGEVYISYPDGVNAPIEYVNACAGPDNDQCHYGSFDLLARVNILKNHCAYLNWIFYCAFNVDKRQMMIDPPRIHKRVEHWSGKFADVEFSERMIYEATYPM

SEQ ID NO: 12 - DNA, 1243 bp, Yarrowia lipolytica
agtgagtattgactggaaagtttgcacaagttcgcaaacacaagattgagattggtataggtccaatgtccactagagccgatacgagctgtctacaattgcctgaatagctcctccatctcgtcaatcgttccgcactaacaaagtgctctcgttaattgccatctttctacttgtcaccacggccctcgctcaaacagccaacatcacgcagtcgacctacgacttcgttctcaagtacggacgactctccaatgtggcctattgtgtcaaggcccccggtccatacgaactggagacggacttcacctgtggaagatcttgcggtcactttcccaacgtgacgttggaacaccagtttggaggcgactttttctccacgtccataaccggctttctggcccacgatcacactaagaaagagaaatacattgttttccgaggcaccttttctctggcagacgccatcaccgacgccttgtttctccaggagccctacctggccgatctgccgcctctgaacaccaccaacatcaactccactagcaattcggcccgagtcgactgccccgattgtgagattcatgacggcttccaaaaggcataccgagagaccatggtgaacatgcagggccacctggtggcctttcttagaaacaacacagactacaagctgatagtcaccggccattctctgggagccgccactgctctgctcatgggcatcaacctgaagaatctcggctttgaccctatggtgatcacgtttggacagcctcgagtgggaaacaaggcgtttgcggactacgccgactcgctctttttcaaacagggagataacggactgaatatcaaccctgaaagacgtctttaccgagtcactcattggaatgacatcgtggtgggagtgcccttctggtccggttatacccatactctgggagaagtgtacatcagctacccagacggagtcaacgcccccatcgaatacgtcaatgcgtgcgctggaccagacaacgaccagtgccactacgggtccttcgacctgctggctcgagtcaacattctcaaaaaccactgtgcctatctcaactggatcttctactgcgcctttaacgtggacaagagacagatgatgattgatcctccgagaatccacaagagagtcgagcattggagcggcaagtttgcagacgtcgagttcagcgagagaatgatctacgaggccacttatcccatgtga

SEQ ID NO: 13 - protein, 363 aa, Yarrowia lipolytica
MLSFIALFLLVAQALAQTAPITQETYDFVLKYGWLSNVAYCVRAPGPFALQSDFTCGNSCAHFPDVTLDYQFGGNFFSTSVTGFLAHDHTKKEKYIVFRGTFSIADAITDIQTIQQPYMTSIPPLNTTDINSTNPSASINCPGCQVHDGFQKAYRETMVNVQDRLVDFLGNNTDYKLIVTGHSLGAVTALFMGINLKNLGYDPTLINYGQPRLGNKAFADYVDALFFKQGDDGLTINPERRMYRVTHWNDFFVGWPAGYSHTLGEVYISDPTGINAPIEDVYSCAGPENNQCHHGSFNLLERLNILKNHCGYLNWIFYCAINVDKREMMIDPPRVGKRVEHWSGKFSDVESTEGLMYEAIYPM

SEQ ID NO: 14 - DNA, 1351 bp, Yarrowia lipolytica
agtgagtatggagagatggactggacttccatcatcgagaagatgtcacaggattgtctgttggcaggaagtctacagctagttgtggtacagtaccattggaagtggcggtggactattaagagttgaagttgttgatgtttctgagaatgcttatcgatcttggaaacgttcaccactgtcagcttatgctcagaacaactcaatgacaacctttccaactcagtacctcagttaattatctcctttgctaatttcagtgctctcgttcattgccctctttcttcttgttgctcaggctctcgcgcaaacggctcctatcacccaggagacatacgactttgttctcaagtacggatggctatctaacgtggcctattgtgtcagagcccctggcccatttgccctgcagagcgattttacctgtggaaactcttgcgctcattttcccgatgtgactcttgactaccagtttggaggaaatttcttctcgacttcagttactggatttttggcccacgatcacaccaaaaaggaaaagtatattgttttccgaggcactttctccattgcagacgctattactgacatccaaacgatccaacaaccctacatgaccagtatccctcctcttaacactactgacataaactccacaaacccttctgccagtatcaattgtcctggctgccaagttcatgatggctttcagaaggcatacagagagactatggtgaacgtacaggatcgtctggtcgacttccttggaaacaacactgactacaaattgatcgtaactggccactctctaggagctgtcactgctctattcatgggcatcaacctgaagaatcttggatatgatccaacgctaatcaactatgggcagcctcgacttggaaacaaggcttttgcagactacgtggacgctctgttctttaagcaaggagacgacggacttaccatcaaccccgaaagacggatgtaccgagtcactcactggaacgacttctttgttggatggcccgctggttattcacatactctgggagaagtttacatcagtgatcccacaggtattaatgctcctattgaagatgtctattcctgcgctggaccagagaataatcagtgtcaccatggatcattcaatcttctagaacgactcaacattctcaagaaccattgcggttatctcaattggattttctactgtgctatcaatgtggacaagagagagatgatgattgatcctccccgagttggcaagagagttgagcattggagtggcaagttttctgatgttgaatcgacagaaggcctgatgtatgaggccatttatcctatgtaa

SEQ ID NO: 15 - protein, 376 aa, Yarrowia lipolytica
MIVLLLLLSVALSSPIMLDRFGDSVNRMIRGNSEAYESGETRGVSQEFEQRLVRYMWFNNVAPCVPKKLQHPFKCIARGCKELGKHTELVDIFTHSDNLFDRTISGFVALDHKHKEIVLALRGTQDAHDWVTDLHLRLVGLHPEHLGVSNFNCRNCQVDLGFLKGYLHSFHVVDSIVQRLTEKYPDYQLVITGHSLGGTAATLFGLNYRLNGYSPLVFSAGAPALGNKQFANFADRVFWGSQNPNTLKVKERDIKFCRMTHLGDFVPRFPFWNGYQQMSGEVFINDVRGIYPPRETLQRCNGQQNRQCSFGDQYRKLEMNFKPHSAYLVPGSECSLSGRRELTFGQVHAANDTNKDSDTNIEPDIEPILIPVVQID

SEQ ID NO: 16 - DNA, 1131 bp, Yarrowia lipolytica
atgattgttctacttcttcttctctctgtcgcgttaagtagtccaattatgttggaccggtttggcgatagtgtgaaccgcatgatacgaggcaacagtgaggcgtatgagagcggcgagacacgcggggtgtcccaggagtttgaacagcggctcgtgcggtacatgtggttcaacaatgtggctccatgtgtccccaagaaacttcagcatccattcaagtgcatagctagaggctgcaaagagctggggaaacatacagagttggtcgacattttcactcattcagacaacttgtttgatcgcacaatttcaggatttgtggctttggatcacaagcacaaggagattgttctggcgttgagaggcactcaggatgcccacgactgggtgacagacctgcatctgaggctagtggggcttcatcccgagcatctgggtgtatcaaacttcaactgtcgcaactgccaggtagacctgggctttctcaagggatatttgcattcgttccatgttgttgacagtattgttcagcgtctgactgaaaaataccccgactaccaactggtgattacgggccattcacttggaggcactgcagctacgctctttggactcaactaccgactcaacggctattctcccctagttttttcggctggagctccggcactgggaaacaagcagtttgccaactttgccgaccgagtcttctggggatcgcagaaccccaacactctcaaggtcaaggagagagatatcaagttctgtcgcatgacccatctgggtgactttgtgcctcggtttcccttctggaacgggtaccagcaaatgtctggagaggtatttatcaacgacgtgcgaggtatatatccacccagggagacactccagcgatgcaacgggcaacagaatagacagtgctcgttcggagaccagtaccgcaagctggaaatgaacttcaagccccactcggcgtacttggtgcctggaagcgagtgttcgctcagtggtaggagagagttgacattcggacaagttcatgcagcgaatgacacaaacaaggactcagacacaaacattgagcctgacattgaaccaatcttgatacctgtcgtgcaaatagactaa

SEQ ID NO: 17 - DNA, 14006 bp, Artificial Sequence
  plasmid containing expression systems for DrBCO, LmATF-S480Q_G409A_V407I_H69A_I484L, and FfRDH
gcggagcctaggccggccagcgtggcgcgtggccatattggccagcagcttactacagctcagccaaagtggatcatgatggacggaaatatcggactggaggccaaaaaggaggtgctcaaatacgcacgggacaagtctgcacaggtggcattcgaacccacgtctgtccccaaagccgctgctctttccgagctcaatctgcccgtttaccccaacaactccattgccttagccacccccaacactgcagagctcgaggccatgttcgaggcattccacgagaagggccgatttgacgttgacgactggtttccagtcatagacggtcttgctctcggagctgactttagaaacggagccaccatgttgtcgcaccaacatcgtggactcaaagctattctcgaacaaggaactctggcccaggcaatccacatgctaccatacatcccaactttgatcatcaagggcggagccaacggcgtcgtcgtcttccaactcattgatgatatcgagtctgccattcattcacagcgttctgcttccaataaaacgcctggcctgttccagaagggcaacgctgcgagtggaaacaccaaggtcggcgtctacatgcagtactttgagcccgaagaagtgggcagtcagtcgattgtaagcgtgactggagctggagacactctgtttggaaccctggccatggagattgtcaaggacgagtcctggttgaacgatatgggagacaagaagagtgcagttgtttctcgagccatgaacaatgctgtgaagactattcagagtaaggacgctgtctgcaagagcatcctttaagtgatttgccatgctttctcttcttccacgatgtaaatacttattttacacactactgtgcagtagcaaatacagaacaagagttgtcgcctattgacagtacagtacgagtagtgtatgtacagtagttatacaatatctatgtgaaattcgtcggcagctttcggctgatgaactacgagttcttcgtaataaatcatcaacgtaataagcttggtaccagagacgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcgctagccacaaaaatgggtgatctcgatgcccgaggaacctctgctcaccccgagctctctgagcgaccttctattatgccttctatgtctgatattcaggacccttctggtgatgacaaggctactccccgaggatctgctgctggtctgccccagcttgagcttgctggacacgcccgacgacttggccaccttgagaacttctttgctgtccaggctcgacagcagatttactcttcttttgctgttttttgtgagtttgacactgcttgttctctcgctcagcttgcttctgctgtgcgaaacgtttgtctttctaacccccttctccttcacactgttgagcctaagcaccctgacatcgctggattctaccactctgacgagtacctttcccgaccttggcctcagcacgattacatgcgagttcttcgagaggttcacgtcgctgacgttgttatgaacggacagaaggagcacgctcacgttgttcgagatgctgttgacgtttttcaggctcacggaaaccaggttacttctgagctccttgagcttatgactcagattgagattcctcacgcttctcagactcgaccctcttggcgacttctctgttttccccacggagaggctaaccgatggcgaacctttgcttttgtttctaaccactgttcttctgatggtctttctggtcttaacttctttcgagatctccagaaggagcttgctcacggccccacctctggtgctcctggtgcccccggagcttccggagttattttcgattacgctcaggacgctgctaccctgcccaagctgccccctcccattgatcagaagctcgattaccgaccttctaagaaggctcttctcggccttctcgctggcaagttcgttcgagagaagctcggttacgtttctgctgctcctcccactacccctacctctgaccttgctcaccctgagggtcaccagtactactgttaccttgttaacgttcccacttcttctgttgcccacattaagactcaggtgcgagagaacgttcctcacaagtgtactctcactccctttctccaggcttgttggcttgtttctctgttcaagtacggtcgagttttttctggttcttggcttgagcgatacaccgatgttcttgttgctatgaacactcgacagcttctccccgaggaccttgagcttcagcgacagtaccgatacggttctaacattggagctgttcgatacaactaccctattgctccccttgacgttcgagataacgatcagaagttctggtcccttgttgagtcttaccgacttgccctttctgatgcccgagataagaacgattacctttacgctcttggtgctcttatgctccctgagatttacgagaagaagaacgttgatgctgttgttaacgataccattcttaaccagcgacgacagggaacccttctttctaacgttggttacgttcgagatgagcagcccactgcttttgctattaagaaccacgttttttctcagggagttggagctaaccgaaacgcttttgttcttaacatttgtgctaccgatcagggtggtcttaacatcgctatttctattgctaagggaacccttgcttctcgacaggagggacaggagctttgtgatatttttaagtctactctccttcgattttaaacgcgtctatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatctgtacctgctgtggaccacgcacggcggaacgtaccgtacaaatattttcttgctcacatgactctctctcggccgcgcacgccggtggcaaattgctcttgcattggctctgtctctagacgtccaaaccgtccaaagtggcagggtgacgtgatgcgacgcacgaaggagatggcccggtggcgaggaaccggacacggcgagccggcgggaaaaaaggcggaaaacgaaaagcgaagggcacaatctgacggtgcggctgccaccaacccaaggaggctattttgggtcgctttccatttcacattcgccctcaatggccactttgcggtggtgaacatggtttctgaaacaaccccccagaattagagtatattgatgtgtttaagattgggttgctatttggccattgtgggggagggtagcgacgtggaggacattccagggcgaattgagcctagaaagtggtaccattccaaccgtctcagtcgtccgaattgatcgctataactatcacctctctcacatgtctacttccccaaccaacatccccaacctcccccacactaaagttcacgccaataatgtaggcactctttctgggtgtgggacagcagagcaatacggaggggagattacacaacgagccacaattggggagatggtagccatctcactcgacccgtcgacttttggcaacgctcaattacccaccaaatttgggctggagttgaggggaccgtgttccagcgctgtaggaccagcaacacacacggtatcaacagcaaccaacgcccccgctaatgcacccagtactgcgcaggtgtgggccaggtgcgttccagatgcgagttggcgaaccctaagccgacagtgtactttttgggacgggcagtagcaatcgtgggcgtagaccccggtgtatataaaggggtggagaggacggattattagcaccaacacacacacttatactacatgctagccacaaaaatgctctctttcttctggcgaaacggtatcgagactcccgagcccctcaaggctgacgtttccggctctatccctccctggcttcagggaacccttctccgaaacggtcctggtctgttctccgttggcaacacttcctacaagcactggttcgatggtatggctctcattcactccttcacctttaaggatggtgaggttttttaccgatctaagtacctgaagtctgagacttacaagaagaacatcgctgccgaccgaatcgttgtgtctgagttcggaaccatggtgtaccccgatccctgcaagaacattttctcccgagccttctcttacatgatgaacgccattcctgactttaccgataacaacctcattaacatcattaagtacggtgaggattactacgcctcctctgaggtcaactacatcaaccagattgaccccctgacccttgagactctcggacgaactaactaccgaaaccacattgccatcaaccttgccactgctcaccctcactacgacgaggagggtaacacttacaacatgggcactgctattatgaacctcggtcgacccaagtacgtgattttcaaggtgcccgccaacacctctgataaggagaacaagaagcctgccctctctgaggtggagcaggtttgctccattcccatccgaccctccctttacccttcttacttccactcttttggcatgactgagaactacatcatcttcgttgagcaggccttcaagctggacatcgtcaagctggctactgcttacttccgagatattaactggggatcttgccttaagttcgaccaggatgacattaacgtgttccacctggtcaacaagaagactggtaaggctgtgtccgtgaagtactacactgacccctttgttaccttccaccacatcaacgcttacgaggacgatggccacgtcgtcttcgatctcattacttacaaggactctaagctgtacgatatgttctacattcagaacatgaagcaggacgtcaagcgatttattgagactaacaaggacttcgctcagcccgtgtgccagcgatttgtccttcccgtcaacgttgataaggagactcctcaggacatcaaccttgtcaagctgcaggacaccactgccactgctgtcctgaaggaggacggctctgtctactgcacccctgacatcatttttaagggtcttgagctccctgctatcaactacaagtttaactctaagaagaaccgatacttctacggcacccgagtggagtggtccccttaccctaacaaggtcgctaaggtggacgttgttactcgaacccacaagatttggactgaggaggagtgttacccttctgagcctgtctttattgcctcccctgacgccgttgatgaggatgacggtgtgattctttcttctgtggtttctttcaacccccagcgaccccctttcctggttgtcctcgatgctaagtccttcaaggagattgctcgagctaccatcgatgcctctattcacatggaccttcacggccttttcatccacgacaagtctacctaagttttttgatcaatgatccaatggctttcacatacccccccacgcctataattaaaacacagagaaatataatctaacttaataaatattacggagaatctttcgagtgttcagcagaaatatagccattgtaacaaaagccggctatcgaccgctttatcgaagaatatttcccgccccccagtggccaaacgatatcggtcagaaggggcagctctaaacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacatgctagccacaaaaatgaccactaagtacacttccgttcacgagtctcccaacggccctggtgacgctcgacccaccgcttcccagattatcgacgattacaaccttgagggagagctttctggcaagactgttctcgtcaccggctgttcctctggtattggtgttgagactgcccgagctatttaccgaactggtgccaccctttacctcactgcccgagatgtcgataaggccaagaccgttcttcccgaccttgttgacacttcccgagtccactttctccaccttgaccttaactctctggagtctgttcgaggttttgctgagaacttcaagtctaagtccactcagcttcacattctcatcgagaacgctggcgtgatggcctgtcccgagggccgaaccgtcgatggttttgagactcagtttggtatcaaccaccttgctcactttctcctcttttacctcctcaaggatacccttctcaactcttctacccccgctttcaactcccgagttgtcatcctctcttcttgtgctcaccaggctggttccgttcaccttaacaacctgtctcttgagggtggatacgagccttggaagtcttacggccagtccaagactgccaacctttggactgcccgagagatcgagaagcgatttggtgcttccggtatccactcttgggctgttcaccccggttccatcgctactgagcttcagcgacacgtttccgacgagcttaagcagaagtgggctgacgataaggagggtgccaagctgtggaagtccaccgagcagggtgccgccaccactgtccttgctgctgtttcccctgagcttgagggtaagggcggtctttaccttgaggatacccaggttgccaagccccctgcccgaggaatgtttggtgttgctgactgggcttacgatgaggatggcccctctaagctctgggccaagtctcttgagctccttaagctccagtaaaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacacactagtggtgtgttctgtggagcattctcacttttggtaaacgacattgcttcaagtgcagcggaatcaaaaagtataaagtgggcagcgagtatacctgtacagactgtaggcgataactcaatccaattaccccccacaacatgactggccaaactgatctcaagactttattgaaatcagcaacaccgattctcaatgaaggcacatacttcttctgcaacattcacttgacgcctaaagttggtgagaaatggaccgacaagacatattctgctatccacggactgttgcctgtgtcggtggctacaatacgtgagtcagaagggctgacggtggtggttcccaaggaaaaggtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggcactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacacctcgagcatgcacactattatcacattactacatccaaacccctacagggggaggagctctccaatcaaatatgtacattaactatctctcgtaaatcattgttataagaccgtccgtgactgtctaaatcggttcattcgttgtaacaaatcagtgtaacaactcgtggtacgcccggtttgcttcgctcacgctcccctagattttccgtctaggacacaacaagccctggtggatgacgttcaacgccttcagcgcgtctttttcctcaataacacacgagatgttaatttcgttggctccctgagaaatcatctcaatgttaatacctgcctgggccagagtagagaagaacatgcctgcacatccgaccatggccttcattctcgttccgatcagcgacacaatggtcatgcctcgcttcacatctactgtgccgtactttcgcagctcctccacagcctgcttgaggttggagtcgggagcatggaaggccatcgacacatggacctcagaagtggagatgagatcgacgacaagtttctgctggtcgagagtagcaaagatcttgttcaagaagccatgactcttggttcgcttattcgaatgaacgttgagaacggtgatattcgatttggtagtgacagctgtaggcttcttctcgtcatctacaggagcagacacctggggagtgaatgagccagaggacagagaagttagcgagtcggtggaagaagccagatcctggacattgtttttcgttttcaggttgcccgagccgtcagcagatgggtagatgatagttcctcctcccagggggttttccacgtttttgattcggataggaatatgggccttaatgacctgctccatggtgaaggggtggatgacttcagatccgtagtaggtcaactcagcggcctcctcgggggtaatgatgggcagcagacgggcagtggacacctttctgggatcggcagtgaagacaccatcgacttctttccagatctgaagctccttggcgtccagacccacagccaccagagcagcacacagatcggtgtatcctcgtccgatctggcttaggaggcctcccttgacggggccaaagaaaccggtcaacacgggcaccatgtttcccttgacaggagcagagtccgtgttaggagagccgggaagagtgatgatctcgcccagaacacgtccgaggtccgaatagaacttgggatcttcggcattggtggtggtaactgcgtgagacaggtcaaagtaccgtgcgttaacaccagcgtcccgcataactgcagtcatgtacatgcaactgagcttctccccaatggccatgatggagtcgagtgttcggggggagatctcagaaatgatttcagcagcagccaggattcgcaacagctggtcgcactcgccgttgatgtcggcattgaggttttcgagcagttcggggttcttgacgtcacgcttagcagctgcaagatggtcttctcggatagactcaatgatggggttgtaggcgtctgatccgagaagagcagagtcagcagcagcaataagacgggtggtggttccctcggccttggtggcctctagatggcctccttggccccattccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgag

SEQ ID NO: 18 - protein, 559 aa, Lachancea mirantina
MGDLDARGTSAHPELSERPSIMPSMSDIQDPSGDDKATPRGSAAGLPQLELAGHARRLGHLENFFAVQHRQQIYSSFAVFCEFDTACSLAQLASAVRNVCLSNPLLLHTVEPKHPDIAGFYHSDEYLSRPWPQHDYMRVLREVHVADVVMNGQKEHAHVVRDAVDVFQAHGNQVTSELLELMTQIEIPHASQTRPSWRLLCFPHGEANRWRTFAFVSNHCSSDGLSGLNFFRDLQKELAHGPTSGAPGAPGASGVIFDYAQDAATLPKLPPPIDQKLDYRPSKKALLGLLAGKFVREKLGYVSAAPPTTPTSDLAHPEGHQYYCYLVNVPTSSVAHIKTQVRENVPHKCTLTPFLQACWLVSLFKYGRVFSGSWLERYTDVLVAMNTRQLLPEDLELQRQYRYGSNVGGVRYNYPIAPLDVRDNDQKFWSLVESYRLALSDARDKNDYLYALGALMLPEIYEKKNVDAVVNDTILNQRRSGTLISNVGYVRDEQPTAFAIKNHVFSQGVGANRNAFVLNICATDQGGLNIAISIAKGTLASRQEGQELCDIFKSTLLRF